(19) United States
(12) Patent Application Publication
(10) Pub. No.: US 2026/0145316 A1
(43) Pub. Date: May 28, 2026

(54) SOFT ACTIVE SKIN FOR STEERING EVERTING ROBOTS

(71) Applicant: THE REGENTS OF THE UNIVERSITY OF CALIFORNIA, Oakland, CA (US)

(72) Inventors: Sukjun Kim, San Diego, CA (US); Tania K. Morimoto, San Diego, CA (US); Shengqiang Cai, San Diego, CA (US); Gaoweiang Dong, San Gabriel, CA (US)

(21) Appl. No.: 18/957,260

(22) Filed: Nov. 22, 2024

(51) Int. Cl. B25J 9/06; A61M 25/00; A61M 25/01

(52) U.S. Cl. CPC B25J 9/065; A61M 25/0012; A61M 25/0045; A61M 25/0113; A61M 25/0155; A61M 25/0158

(57) ABSTRACT

A steerable device and methods of navigating and manufacturing the steerable device are described. An example method of navigating the steerable device includes selecting one or more actuators from a plurality of actuators positioned at a plurality of locations on a flexible skin of the steerable device, allowing a gas or fluid to be directed to a hollow interior of the flexible skin to form an elongate body, and activating the one or more actuators by applying heat to the one or more actuators to cause the one or more actuators to contract and to thereby bend the elongate body at a particular angle. The one or more actuators are made of a flexible material, and are selected to, upon activation, bend one or more sections of the flexible skin at one or more angles.

### STATEMENT REGARDING FEDERALLY SPONSORED RESEARCH

This invention was made with government support under grant number 13499720 awarded by the National Institutes of Health (NIH). The U.S. government has certain rights in the invention.

### TECHNICAL FIELD

This document relates to a steerable device and methods of navigating and manufacturing the steerable device.

### BACKGROUND

Soft robots are well-suited for navigation through delicate and constrained environments due to their ability to leverage their structural compliance to adapt and interact safely with their surroundings. Soft growing robots, or vine robots, in particular, have demonstrated their ability to traverse a wide range of complex environments. Using the inherent compliance of the body, the robot can follow a constrained path, such as a pipe, and can passively bend or buckle to conform to the environment. However, navigating through environments with more complicated constraints, such as branches with high tortuosity, requires integrated mechanisms to actively steer the robot in a desired direction.

### SUMMARY

The disclosed technologies provide steerable soft growing robots by integrating flexible actuators (such as liquid crystal elastomer (LCE) actuators) and flexible heaters into the soft robot skin and demonstrate their unique ability to navigate tortuous paths with multiple bends at the small (e.g., millimeter) scale. In some examples disclosed herein, the heaters may be referred to as thermal elements, and the steerable soft growing robots may be referred to as steerable devices. Additionally, the terms robot or robot arm are used in this patent document to describe some of the example implementations, but it is understood that the disclose technology relates to various types of steerable devices that can navigate tortuous path in accordance with the disclosed embodiments.

Soft growing robots move forward by extending their body from the tip via eversion, which allows for minimal interaction with the environment. When integrated with active steering mechanisms, these robots can control the direction of growth, enabling them to traverse complex and highly tortuous environments. Using existing active steering strategies, however, it is challenging to miniaturize these robots and to achieve multiple bends while keeping the robot body fully soft. The disclosed technologies describe millimeter-scale, steerable, and fully-soft growing robots by functionalizing the robot skin with flexible actuators. Some example materials for the flexible actuators include LCE and/or shape memory polymers. Based on the disclosed technology, small-scale (e.g., less than 5 mm diameter) robots can achieve a wide range of bending angles (e.g., greater than) 100° at multiple points along their length.

An example scaling analysis shows the effects of the control inputs and design parameters on the achievable bending angles, and helps to guide the design and control of an example robot. In an example embodiment, robot's steering capability is demonstrated by embedding six flexible heaters into the robot skin and navigating tortuous paths with multiple turns. Please note although six flexible heaters are embedded into the robot skin, the number of the embedded flexible heaters is not limiting. The example robot can steer even after squeezing through a narrow gap that is 50% of the robot's diameter by leveraging its fully-soft body. The disclosed technologies find many applications that include, among others, minimally invasive surgical procedures, inspection of components, and generally monitoring, imaging or other procedures in environments that requires small-scale and flexible devices that can navigate tortuous paths. The example results highlight the advantages of functionalizing the skin of soft growing robots that enables small, steerable, and fully-soft growing robots to be used in delicate and constrained environments.

An aspect of the disclosed technology relates to a steerable device. The steerable device includes an elongate body comprising a flexible skin that surrounds a hollow interior and a plurality of actuators positioned at a plurality of locations of the flexible skin. Each of the plurality of actuators includes a material that selectively contracts or expands in response to a change in temperature. The steerable device further includes a plurality of thermal elements coupled to the plurality of actuators, each of the plurality of thermal elements operable to change a temperature of a respective actuator to cause the respective actuator to contract or expand. The hollow interior is configured to receive a gas or fluid to allow expansion of the flexible skin along a longitudinal direction. The elongate body is configured to be expanded along the longitudinal direction in response to a flow of the gas or fluid inside the hollow interior of the flexible skin and to be bent at one or more angles in response to at least a temperature change of one or more of the plurality of actuators.

Another aspect of the disclosed technology relates to a method of navigating a steerable device. The method includes selecting one or more actuators from a plurality of actuators positioned at a plurality of locations on a flexible skin of the steerable device. The one or more actuators are made of a flexible material, and are selected to, upon activation, bend one or more sections of the flexible skin at one or more angles. The method further includes allowing a gas or a fluid to be directed to a hollow interior of the flexible skin to form an elongate body and activating the one or more actuators by applying heat to the one or more actuators to cause the one or more actuators to contract and to thereby bend the elongate body at a particular angle.

A further aspect of the disclosed technology relates to a method of manufacturing a steerable device. The method includes providing an assembly by performing a first thermal processing for an inner skin and an outer skin at a first temperature for a first period of time to allow for encapsulation of one or more heaters within the inner skin and the outer skin, folding the assembly to form a tube, performing a second thermal processing for the tube at a second temperature for a second period of time, and inserting one or more actuators between the outer skin and a plurality of straps and in thermal contact with the one or more heaters after the second thermal processing. Each of the one or more actuators is fixed at both ends onto the outer skin. The method further includes inverting a first end of the tube inside.

### BRIEF DESCRIPTION OF THE DRAWINGS

FIG. 1 shows an example steerable soft growing robot according to an embodiment of the present disclosure.

FIG. 2 shows an example modeling and scaling analysis of a steering angle according to an embodiment of the present disclosure.

FIG. 3 shows example steering characterization results according to an embodiment of the present disclosure.

FIG. 4 shows an example application of a robot navigating through various way points along different paths according to an embodiment of the present disclosure.

FIG. 5 shows an example application of a robot navigating an aortic arch model and delivering catheter to arteries according to an embodiment of the present disclosure.

FIG. 6 shows an example application of a robot navigating a mock jet engine model and demonstrating inspection according to an embodiment of the present disclosure.

FIG. 7 shows an example fabrication process for an LCE-driven steerable soft growing robot according to an embodiment of the present disclosure.

FIG. 8 shows a schematic diagram of a circuit used for controlling a pressure inside the hollow interior and temperatures of a plurality of actuators according to an embodiment of the present technology.

FIG. 9 shows a flowchart of implementing a method of navigation a steerable device according to an embodiment of the present disclosure.

FIG. 10 shows a flowchart of implementing a method of manufacturing a steerable device according to an embodiment of the present disclosure.

### DETAILED DESCRIPTION

Two main categories of active steering approaches have emerged for this class of robot: distributed steering, which bends a significant length of the robot body, and localized steering. To achieve active distributed steering, pneumatic artificial muscles (PAMs) and tendon-driven mechanisms have been integrated along the robot length. When actuated, one side of the robot body changes in length, resulting in constant curvature bending of the entire body. While PAMs enable fully-soft steerable growing robots, the amount of actuation strain is limited, making it difficult to achieve high curvatures. Tendon-driven actuation, on the other hand, can achieve tight curvatures in a more precise manner, but the friction between the tendon and the robot limits the robot length, and often rigid components such as tendon stoppers are required. In addition, the integration of both PAMs and tendon-driven mechanisms becomes more difficult on a small scale, motivating the recent development of a steerable soft growing robot with magnetized skin. The growth direction of the robot was controlled by changing the external magnetic field using a permanent magnet. Although this approach enables miniaturization of the robot body, the small magnetic volume in the coating on the robot skin necessitates a strong magnetic field for steering, which ultimately limits the scalability of the system. In general, one of the challenges with active distributed steering approaches is that the actuation deforms the entire body, making the steering angle dependent on the robot length and interaction between the robot body and the environment. While selective actuation of PAMs has enabled more localized steering and shape control of the robot, it comes at the cost of a significant increase in design complexity.

The other main category of active steering is localized steering mechanisms, which allow the robot to decouple the steering angle from the robot length and shape. Several localized steering mechanisms have been developed, typically relying on the addition of an electromechanical device that sits at the tip of the robot. Many of these devices are linkage-based mechanisms integrated inside the robot, and they are articulated by either a motor, tendons, or pneumatic actuators. Another approach has been to use a heat-welding mechanism on the outside of the robot to selectively deform the robot body by heat-welding certain points of the thermoplastic body, but the process is irreversible. Because these localized steering approaches all rely on bulky and rigid electromechanical devices, the robot cannot remain fully soft and is limited in its ability to be miniaturized.

The disclosed technologies present example small scale, steerable, and fully-soft growing robots that can navigate through highly tortuous paths and tight spaces. In some example embodiments, robot design and manufacturing process functionalize the soft robot skin with LCE actuators that contract when heated, causing the robot to bend at that location. Compared to existing designs, the disclosed technologies, among other features and benefits, enable growing robots to achieve high-curvature, localized steering at multiple points along their length.

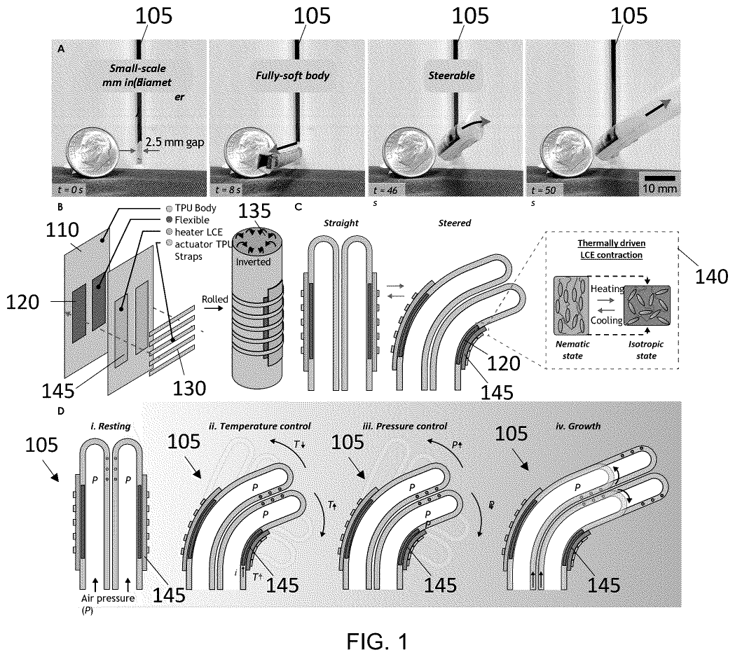

FIG. 1 shows an example steerable soft growing robot 105 according to an embodiment of the present disclosure. In some examples, the steerable soft growing robot 105 is referred to as a steerable device. The steerable soft growing robot 105 in FIG. 1 includes LCE actuators 145, and accordingly can be referred to as an LCE-driven steerable soft growing robot. Alternatively or additionally, the steerable soft growing robot 105 may include one or more actuators that are made of other materials, such as shape memory polymer. FIG. 1(A) shows an example small-scale (5 mm in diameter), steerable, and fully-soft growing robot 105. FIG. 1(B) shows the robot 105 includes a Thermoplastic polyurethane (TPU) body 110, embedded flexible heaters 120, and the LCE actuators 145. The stacked film 135 is rolled into a tube and inverted at the tip to form the robot body 110. FIG. 1(C) shows a cross section of the robot 105 in the straight and steered configuration. The inset 140 shows the principle of LCE actuation, where the phase of the LCE changes when heated, causing contraction. FIG. 1(D) shows an example steering strategy: i) The robot body 110 is an inflated thin-walled tube with an embedded set of LCE actuators 145 and heaters 120 in the resting state. ii) The LCE actuator 145 on one side contracts when heated, enabling the robot 105 to steer. Flexible heaters 120 embedded in the skin of the robot 105 deliver heat to the LCE actuators 145 through, for example, Joule heating. In some embodiments, by controlling the temperature, the steering angle can be changed. iii) While the actuator is activated, internal pressure can be controlled to change the steering angle. iv) While keeping the steering angle, the robot 105 can grow from the tip.

The present technologies characterized the new robotic system, including performing a scaling analysis, in order to validate the effectiveness of using LCE actuation for steering soft growing robots at small scales. The present disclosure also studies how the design parameters and control inputs (temperature and pressure) affect the steering performance. Example results show that a 5 mm diameter robot was able to achieve a wide range of steering angles (>) 100° by heating the LCE to 65° C. and using the internal pressure of the robot as the primary control input for steering.

The disclosed robot architecture and thorough characterization studies enabled the robot to demonstrate its ability to navigate through tortuous paths and narrow gaps. For example, a robot with six integrated heaters and actuators is able to navigate through four different paths that included multiple, high-curvature turns. In addition, by leveraging the functionalized soft skin of the robot with flexible LCE actuators and heaters, the robot 105 was able to maintain its fully soft body, demonstrating the ability to steer even after squeezing through a gap that was 50% of the robot's diameter as shown in FIG. 1(A). Finally, the heaters 120 and actuators 145 can be manufactured and integrated at different locations along the robot 105 to successfully demonstrate real-world applications, such as navigating through a model of the aortic arch for medical procedures and through a jet engine model for critical inspection tasks. As disclosed herein, functionalizing the skin of soft growing robots 105 with LCE actuators 145 provide several advantages that include enabling small-scale, steerable, and fully-soft robots with potential for applications in delicate and constrained environments.

Steerable Soft Growing Robot with LCE Actuators: Principles and Designs

The ability to steer across a wide range of angles is important for enabling navigation through unstructured, complex environments. One example small-scale, steerable soft growing robot 105 with LCE actuators 145 and flexible resistive heaters 120 is illustrated in FIG. 1.

LCEs are polymer networks that incorporate liquid crystal mesogens into the polymer chains. As the temperature rises, LCEs transition from a nematic phase to an isotropic phase, resulting in significant and reversible deformation. Thanks to the thermally induced deformation of LCEs, they have been extensively explored as artificial muscles for constructing soft robots of various forms, such as LCE-fiber based tensegrity robots, swimming robots, crawling robot, and legged robot. Some advantages of the LCEs include large work density with high actuation stress and strain, ease of fabrication, high stretchability and compliance, excellent tunability of their thermo-mechanical properties, and even great biocompatibility.

By strategically integrating multiple LCE actuators 145 around the circumference and along the length, the robot 105 can steer in various directions at multiple locations. In the example, two sets of actuators and heaters are embedded and located 180° part, into the skin of the robot to enable planar bending as shown in FIG. 1(D)-i. 3D steering can be enabled by evenly arranging three or more sets of actuators and heaters around the circumference of the robot. The inverted tail of the robot is held in position at the base to prevent undesired growth, and the robot is pressurized with air. It should be noted that air is one non-limiting example of a gas that can be used to pressurize the robot arm. It should be further noted that in some embodiments, a fluid can be used to allow the robot arm to inflate. When one of the heaters is Joule-heated by applying current (i), the LCE actuator 145 on the same side heats up and contracts, bending the robot body 110. Depending on the temperature of the LCE (T), different bending angles can be achieved. If the temperature is higher, the amount of actuation stress and strain the LCE can generate is higher, resulting in a larger bending angle, and vice versa as shown in FIG. 1(D)-ii. Alternatively, the pressure (P) inside the robot 105 can be used to change the bending angle. While keeping the temperature on the LCE actuator constant, a lower pressure can result in a larger bending angle since it reduces the effective stiffness of the robot as shown in FIG. 1(D)-iii. After the robot 105 is bent to a desired angle, the robot 105 can grow via eversion of the material at the tip as shown in FIG. 1(D)-iv. For the robot 105 to grow while steered, the pressure has to be high enough to evert the material at the tip without affecting the bent shape of the robot 105.

Because the skin of soft growing robots experiences extremely high deformation during the eversion process, any integrated elements, including actuators and heaters, must be thin, flexible, and robust to avoid mechanical or electrical failures. To address this challenge and create a functionalized skin, we utilized thin LCE actuator strips and thin-film flexible heaters made of electrically conductive polymer, PEDOT:PSS. As seen in FIG. 1(B), the flexible heaters 120 and LCE actuators 145 are embedded into different layers of the robot skin. The flexible heaters 120, which serve to transfer heat to the LCE actuator 145 via, for example, Joule heating, are embedded into the inner skin. The LCE actuators 145 are attached at both ends to the outer skin of the robot 105, and thin straps 130 are then wrapped around the LCE actuators 145 to mechanically constrain them to the robot body 110. These straps 130 help to ensure that the LCE actuators 145 maintain good contact with the embedded heaters 120 and enable more constant curvature bending when the LCE actuators 145 are heated and contracted along the length as shown in FIG. 1(C). Detailed manufacturing processes are described in greater details below.

### Example Modeling and Scaling Analysis

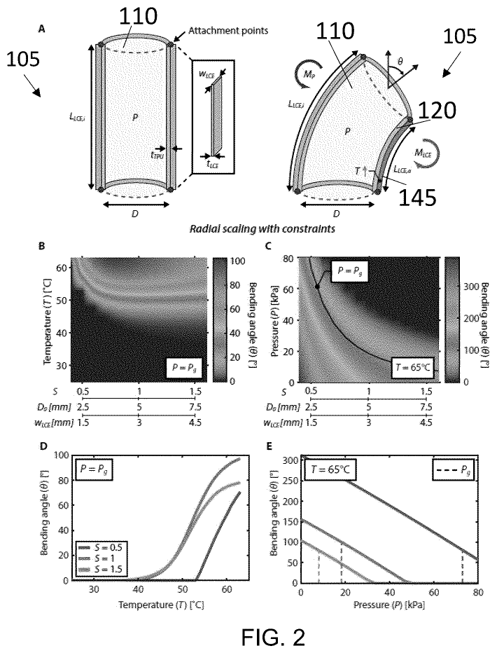

FIG. 2 shows an example modeling and scaling analysis of a steering angle according to an embodiment of the present disclosure. FIG. 2(A) shows a cross section of the robot 105 and relevant design parameters for the model. When an LCE actuator 145 on one side of the robot 105 is heated and contracts, the inflated tube bends to an angle (θ). FIG. 2(B) shows a radial-scaling analysis of the bending angle (θ) when changing the temperature (T) of the LCE actuators 145. FIG. 2(C) shows a radial-scaling analysis of the bending angle (θ) when changing the pressure (P) of the robot 105. The length (L_{LCE,i}) and thickness (t_{LCE}) of the actuator 145, and the thickness of the body (t_{TPU}) are kept constant. S is a scaling constant multiplied by the design parameters (D and w_{LCE}). (D) Bending angle (θ) when radially scaling the robot with the same design constraints as above with respect to temperature (T), (E) and pressure (P) at S=0.5, 1, and 1.5.

In the examples below, the bending of the soft growing robot 105 via LCE actuation is modeled to illustrate (i) the effectiveness of each control input (pressure and temperature) and (ii) the influence of scaling the robot 105. FIG. 2(A) shows the cross section of the robot 105 before and after LCE actuation, along with the critical design parameters. The disclosed technologies make the following assumptions in the modeling. First, the disclosed technologies assumes that the robot body 110 is an inflated thin-walled tube with diameter D, and the disclosed technologies considers only a segment of the robot 105 with length L_{LCE,i} where LCE actuators 145 are present. The tube was assumed to be stretchable in only the radial direction, such that D changes with the internal pressure (P). Rectangular LCE actuators 145 (w_{LCE}×L_{LCE,i}×t_{LCE}) are attached to the tube at each end, and the actuator 145 is assumed to remain in contact with the tube during actuation. When the LCE actuator 145 on one side is actuated by heating, the length of the actuator 145 contracted to L_{LCE,a} and applied a moment (M_{LCE}), which results in constant-curvature bending. During actuation, the length of the other side of the tube was assumed to remain constant. Due to the internal pressure and the volume change in the robot 105, a resisting moment (MP) was generated, and by solving M_{LCE}+M_{P}=0, the bending angle (θ) could be calculated. The stress-strain relationship of the LCE is assumed to be linear, and the blocked stress and free actuation strain used in the model are fitted from experimental results.

If the robot 105 is scaled isometrically without any design constraints, the present disclosure provides that the bending angle is scale-independent, highlighting the applicability of the disclosed technologies across scales. However, in many practical scenarios, certain design parameters can be difficult to scale and others can be tied to the application-specific design requirements. Therefore, the present disclosure focuses on radially scaling the robot 105 by a scaling factor(S), that spanned from 0.5 to 1.5, under the following set of design constraints.

First, the thickness of the robot body (t_{TPU}) and LCE actuator (t_{LCE}) cannot be arbitrarily scaled in practice due to availability in off-the-shelf materials and manufacturing processes, respectively. Second, the actuator length (L_{LCE,i}) can often be dependent on the application based on the need to achieve a required range of bending angles and curvatures. Therefore, the robot diameter (D) and the actuator width (w_{LCE}) were scaled by S, while the other design parameters (t_{T}PU, t_{LCE}, and L_{LCE,i}) were kept constant in this study.

In FIG. 2(B), the temperature applies to the LCE actuators 145 and the scale of the robot 105 were varied while the pressure was kept at the growth pressure (Pg), which is defined as the minimum pressure required for robot growth. Since robot growth is a function of soft growing robots, and the pressure required for growth depends on the robot size and materials, Pg was used in the scaling analysis, rather than maintaining a constant pressure across scales. The disclosed technologies use a simplified model of Pg that assumed there was no friction between the robot body 110 and the tail at the start of growth. Overall, the example model shows that a wide range of bending angles can be achieved by controlling the temperature of the LCE actuator 145. As expected, a greater bending angle was predicted at a higher temperature due to a larger force and displacement from the actuator 145. We also found that the maximum bending angle could be theoretically achieved at S=0.78. FIG. 2(D) shows the relationship between the temperature and bending angle at three different scales (S=0.5, 1, and 1.5). The θ-T relationship while keeping the pressure at Pg was nonlinear since the LCE actuation performance rapidly varies under the change in temperature.

The pressure was also utilized as a control input while keeping the temperature constant at 65° C. in FIG. 2(C). 65° C. was chosen for the LCE to fully change its phase for actuation. In this case, larger bending angles were predicted at lower pressures due to a reduced resisting moment (MP). As seen in FIG. 2(E), the bending angle showed a linear relationship with pressure. The dashed lines represent Pg at each scale and show that Pg increases significantly when the robot 105 is scaled down (as shown in FIG. 2(C)). Therefore, while a larger range of bending angles can be achieved at the smaller scale with the same range of pressure inputs, the bending angles could be limited by the growth pressure line if the robot 105 has to grow further while maintaining the bending angle.

The example modeling results in FIGS. 2(B)-(E) show how the bending angle can be controlled by changing temperature or pressure. While temperature control allows direct control over the actuators 145 for bending, the θ-T relationship is nonlinear, and only a narrow range of the control input (45° C.˜65° C.) is available, which results in a rapid change in bending angle from a small change in temperature. Pressure control shows a linear θ-P relationship, and a wider range of control inputs can be utilized for bending. Since the robot is a single pressurized tube, one pressure sensor at the base would suffice for precise pressure control. Accordingly, the disclosed technologies used pressure control for achieving various bending angles, while keeping the LCE activated at a single temperature.

TABLE 1
Design parameters used in the scaling analysis in FIG. 2 when
the robot 105 is radially scaled with design constraints.

| | | | |
|---|---|---|---|
| S | 0.5 | 1 | 1.5 |

| | | | | | | |
|---|---|---|---|---|---|---|
| D₀ | 2.5 | mm | 5 | mm | 7.5 | mm |
| w_{LCE} | 1.5 | mm | 3 | mm | 4.5 | mm |
| t_{LCE} | 170 | μm | 170 | μm | 170 | μm |
| L_{LCE, i} | 35 | mm | 35 | mm | 35 | mm |
| t_{TPU} | 50 | μm | 50 | μm | 50 | μm |

### Robot Characterization Examples

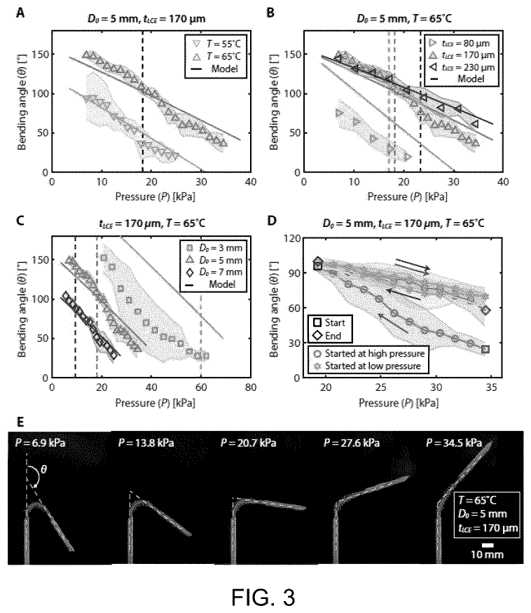

FIG. 3 shows example steering characterization results according to an embodiment of the present disclosure. FIG. 3(A) shows how a steering angle varies with the pressure at different temperatures FIG. 3(B) shows how a steering angle varies with the pressure at different LCE thicknesses. FIG. 3(C) shows how a steering angle varies with the pressure at different robot diameters. FIG. 3(D) shows how a steering angle varies with the pressure when the pressure is modulated and LCE is kept actuated. The shaded region in the example represents the standard deviation (N=3). Vertical dashed lines indicate the growth pressure (Pg) of each robot design. FIG. 3(E) includes still frame images showing that the robot can achieve a wide range of bending angles by controlling the pressure.

The disclosed technology is used to fabricate example soft growing robots that are 250 mm in length with two sets of flexible heaters and LCE actuators located on opposite sides. The length and width of the LCE actuator were 35 mm and 0.6 D₀, respectively, where Do is the nominal diameter of the robot when the pressure is zero. The length of the embedded flexible heater was also 35 mm, and the width was 0.8 D₀, which was slightly wider than the actuator to ensure uniform temperature across the LCE actuator. The steering capabilities are extensively characterized across a wide range of design parameters and control inputs. Using pressure as the primary control input, the effects of temperature, LCE thickness, and the robot diameter on steering angles is evaluated as shown in FIGS. 3(A)-(C). The pressure was first applied, and then the LCE actuator was activated. Between each measurement, the actuator was turned off and reset. We also evaluated the effect of pressure modulation on steering angle while the LCE was actuated as shown in FIG. 3(D). Each data point and the shaded region represent the mean and standard deviation of the measured angles, and overlaid solid lines show the modeled bending angles. The maximum pressure tested for each device was empirically determined to avoid excessive stretch on the robot skin. The dashed vertical lines on the plots represent the growth pressures of the robots (P_{g}), which were experimentally characterized. This information shows that the maximum bending angle is limited by P_{g} if the robot needs to grow further while maintaining the bending angle.

### Illustrative Effects of Temperature

FIG. 3(A) shows the bending angles when the actuator is, for example, Joule heated to 55° C. and 65° C., which are chosen based on the thermo-mechanical properties of the LCE. An electro-thermal characterization of the flexible heaters was conducted to determine the required electrical power to heat the actuator to the desired temperatures. When the actuator was operated at a higher temperature, a greater bending angle was achieved at a given pressure since the LCE actuator could provide larger stress and strain. At 65° C., the robot was able to achieve a wide range of bending angles from 36° to 101° at pressures higher than P_{g}. FIG. 3(E) shows photos of the robot while bent at various pressures. The results under both temperature conditions showed good agreement with the model, with larger variations seen at a lower temperature, which was not surprising considering how rapidly the LCE's actuation stress and strain change around 55° C.

### Illustrative Effects of LCE Thickness

The effect of LCE thickness (t_{LCE}) on bending angle was characterized and results are shown in FIG. 3(B). Three thicknesses, 80, 170, and 230 μm are tested. While thicker LCE actuators (230 μm) can provide larger actuation stress and thus enable greater bending angles, P_{g} also increases significantly resulting in smaller bending angles at P_{g} when compared with the robot with 170 μm thick actuators. The bending angle with the thinnest actuator (80 μm) was substantially smaller than what the model predicted, since the actuation force was not strong enough to overcome the frictional force between the actuator and the robot body.

### Illustrative Effects of Scaling

The robot was radially scaled by changing the nominal diameter of the robot (Do) to 3 mm, 5 mm, and 7 mm to study the effect of scaling on steering performance as shown in FIG. 3(C). Following the same design constraints made in the modeling and scaling analysis, the width of the heater and LCE actuator are scaled while keeping the length and thickness of LCE actuator constant. While the robots with smaller Do are able to achieve larger bending angles at a given pressure, P_{g} also increases significantly. This results in the robot with 5 mm in diameter achieving the largest bending angle at P_{g}, as predicted from the modeling and scaling analysis. When Do was the smallest (3 mm), there were large discrepancies in bending angles between the example model and the example experiments. These phenomena were mainly attributed to the higher ratio of wall thickness to diameter (Do) at smaller scales, since the thickness of the robot body, flexible heaters, and LCE actuators remained the same for the characterization. Added bending stiffness from the wall thickness, which was not considered in the model, resulted in overestimation of the model and an increase in P_{g}.

### Illustrative Effect of Pressure Change

In order to precisely control the bending angle using pressure as the primary control input, fine adjustments of pressure are required. In addition, in order for the robot to grow while bent, the LCE actuators must remain on, while the pressure may need to be increased to overcome the friction between the robot body and the inverted tail. FIG. 3(D) shows an example effect of keeping the LCE actuated as the pressure is changed in two different orders. In a first example, the pressure is decreased from the maximum pressure to the growth pressure (P_{g}) and then was increased back to the maximum pressure as shown in FIG. 3(D). While decreasing the pressure, the bending angle increases linearly from 25 to 95, closely following the trend of the experimental result as shown in FIG. 3(A). However, when the pressure is increased back to the maximum pressure, the change in bending angle is smaller, decreasing from 95 to only 58. This difference can likely be explained by the formation of multiple kinks in the robot skin along its length when the robot achieved a large bending angle. Some of these kinks could not be unkinked when the pressure was increased due to friction between the skin and LCE actuator, resulting in a smaller decrease in the bending angle. In a second example, the pressure is first increased from P_{g} to the maximum pressure, and then was decreased back to P_{g} as shown in FIG. 3(D). When the pressure is increased, the total change in bending angle was small, similar to what was observed when the pressure was increased in the previous case. When the pressure was then decreased, the bending angle followed a similar trajectory almost without any hysteresis. From this characterization, one of the key findings was that the bending angle was dependent on the initial pressure and the direction of the pressure change. In detail, FIG. 3(D) shows that changing the pressure by the same amount, but in different directions, resulted in a different overall change in bending angle. Moreover, FIG. 3(D) shows that the initial pressure determined whether or not hysteresis would be observed. This information can be used to select a pressure modulation strategy to either achieve the desired bending angles without any hysteresis (as suggested by FIG. 3(D)) or to leverage the ability to achieve the same bending angle at different pressures (as suggested by FIG. 3(D)) in order to control both the bending angle and growth of the robot.

### Example Tradeoffs in Robot Design

The soft growing robot is able to steer across a wide range of angles, and grow while steered to navigate through complex environments. The characterization results shown in FIGS. 3(A)-(C) illustrate how the changes in design parameters and control inputs affect the robot performance. First, using pressure as the primary control input resulted in a linear relationship between the control input and bending angle as expected from the model, and a good agreement between the model and experiment was observed. While the temperature of the LCE actuator is hard to control precisely, and the LCE actuation is very sensitive to change in temperature during the phase transition, the pressure inside the robot is easier to measure and control precisely. One downside of pressure control was a limit on the smallest bending angle that could be achieved, since small bending angles require higher pressures, which can cause the robot to radially stretch and ultimately burst. This limitation can be mitigated by using a lower actuation temperature (55° C.) at the cost of larger variations in bending angles (as shown in FIG. 3(A)) since the actuation performance of the LCE can rapidly vary around this temperature.

Second, robots with a smaller diameter or a thicker LCE actuator can achieve greater steering angles with the same pressure inputs. However, we also observed that the growth pressure of these robots increased significantly due to a greater relative wall thickness compared to the robot diameter. Consequently, the maximum bending angle the robots could achieve when pressurized above the growth pressure was smaller. The trade-off between the range of bending angles and the growth pressure needs to be carefully considered in the robot design.

Lastly, we found that a thin LCE actuator or small robot diameter led to larger discrepancies between the model and experiments. Overcoming the friction between the LCE actuators and the robot body was more difficult for thin LCE actuators, and the added stiffness from the wall thickness was more substantial for the robots with small diameter.

With these design trade-offs in mind, different design parameters can be chosen depending on the application and required characteristics, such as the size of the robot and the maximum bending angle. For the remainder of the examples, we mainly used the robot design with D₀ and t_{LCE} of 5 mm and 170 μm, which was able to achieve the largest bending angle at pressures higher than the growth pressure among the tested set of design parameters.

### Robot Application Example—Navigation Through Narrow Gaps

One characteristic of the disclosed steerable devices is that its steering capability is achieved by its soft functionalized skin, which does not require any rigid components to assist with steering, even at the small-scale. This feature enabled the robot to demonstrate steering even after growing through a gap that was smaller than the robot diameter. For example, as shown in FIG. 1(A), the 5 mm diameter robot 105 is able to grow through a small vertical gap with a width that was 50% smaller than the robot diameter. The required pressure to grow through the gap (2.5 mm width) was increased to 28.0±0.4 kPa (N=3), which was 54% greater than the growth pressure in open space (18.2 kPa). A 2 mm wide gap was also tested, however, the robot 105 is not able to grow through the gap with an applied pressure of less than 34.5 kPa. Higher pressure was not tested to avoid excessive stretch on the robot body that can cause failure. After squeezing through the small gap, the robot can demonstrate steering and then growing while steered since the force applied by the gap or reduced volume does not affect the steering capability.

### Robot Application Example—Navigation Through Open Space with Multiple Tortuous Paths

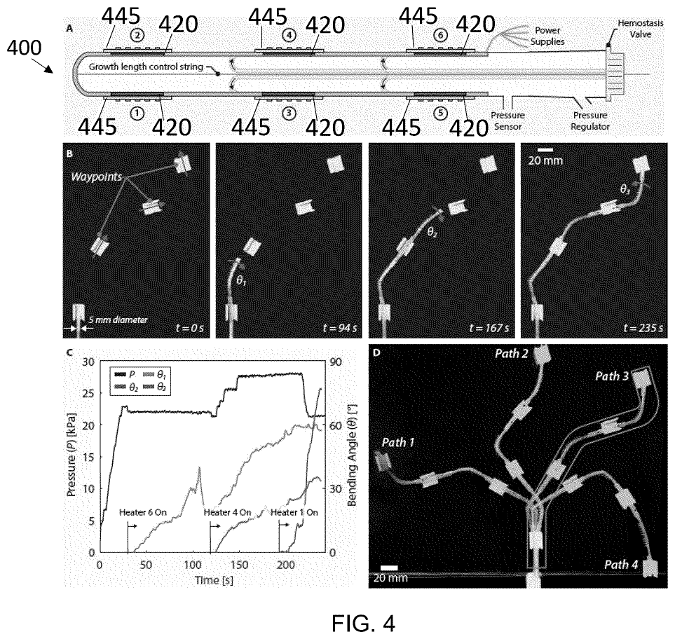

FIG. 4 shows an example application of a robot 400 navigating through various way points along different paths according to an embodiment of the present disclosure. FIG. 4(A) shows a schematic diagram of the robot 400 according to an embodiment of the present disclosure. As shown, six heaters 420 and actuators 445 are integrated into a 300 mm long robot to enable navigation through tortuous paths with multiple bends. However, the numbers of the heaters 420 and respective actuators 445 are not limiting. FIG. 4(B) shows the robot navigating through waypoints by steering at three different locations and growing to reach the end according to an embodiment of the present disclosure. FIG. 4(C) shows an example measured internal pressure of the robot 400 and bending angles. FIG. 4(D) shows the example robot 400 can navigate multiple tortuous paths. As shown in FIG. 4(D), photos for the example four paths are overlaid.

The robot skin can be functionalized to achieve high-curvature and localized steering at multiple locations along the body length and circumference to traverse through a wide range of challenging environments. A 300 mm long, 5 mm diameter robot with six actuators (t_{LCE}=170 μm) and heaters was designed and manufactured as shown in FIG. 4(A) to demonstrate its steering capability in a 2D plane. To control the growth length of the robot, a string was connected at the robot tip and fed through a hemostasis valve for manual control. FIG. 4(B) shows the time series of the robot navigating through a path with 3 waypoints which requires 30, 45, and 90 steering angles sequentially. By selectively activating the integrated actuators, the robot was able to successfully reach the end of the path. The measured internal pressure and bending angles are plotted in FIG. 4(C). Because pressure was used as the primary control input, the bending angles at the proximal end of the robot were affected by the pressure change required for con-trolling the bending angles at more distal end. These changes in the proximal bending angles did not affect the ability of the robot to successfully steer at the tip, and the use of a combined temperature and pressure control scheme could be implemented in future work if maintaining a specific shape for the proximal part of the robot is required for a given application. In addition, the bending angle of proximal sections could also be affected by phenomena such as the applied tension to the tail and the interaction between the robot and the waypoint, as was likely the case from 86 s to 113 s. The growth length control can be automated in the future to mitigate any excessive tail tension. The robot was able to navigate four tortuous paths with different sets of bending angles by selectively activating the heaters along the robot as shown in FIG. 4.

The environment setup in FIG. 4 is built by sandwiching two acrylic plates with 3D-printed waypoints attached in the middle. The gap between the two plates is 8.5 mm, which is 70% larger than the nominal robot diameter. The robot length is controlled manually by holding the growth length control string at the base. For the robot navigating the aortic arch model, a catheter (for example, IC71132UG, Cerenovus) is integrated at the tip, and the catheter was manually held and fed through the hemostasis valve to control the growth length.

### Robot Application Example—Medical Applications

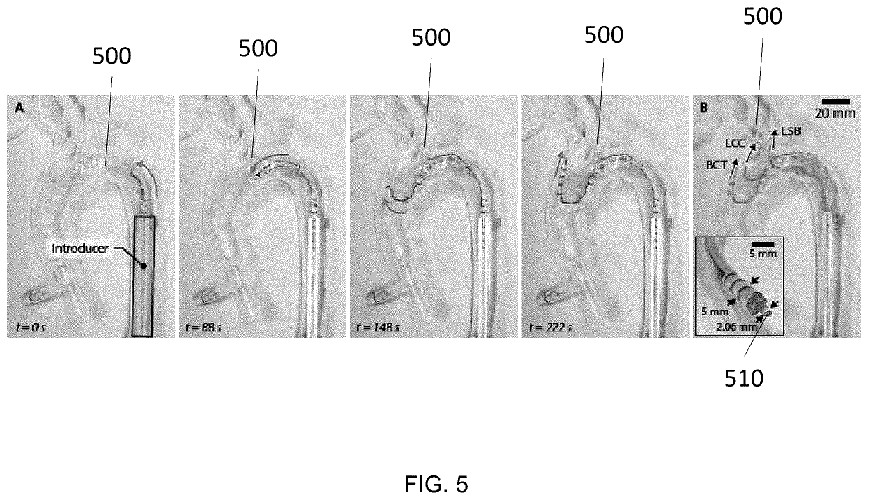

FIG. 5 shows an example application of a robot 500 navigating an aortic arch model and delivering catheter to arteries. Robot 500 navigating an aortic arch model and delivering catheter 510 to arteries. FIG. 5(A) shows example video stills of the robot 500 navigating the aortic arch and accessing the brachiocephalic artery (BCT). FIG. 5(B) shows example photos from three different trials are overlaid showing the robot 500 accessing all three arteries from the aortic arch. The inset in FIG. 5(B) shows the catheter 510 integrated with the soft growing robot 500. The outer diameter of the catheter 510 was 2.06 mm.

Specifically soft growing robots with a small diameter and steering capabilities have significant potential for use in a wide range of applications. To illustrate one example, FIG. 5 in the present disclosure demonstrates an example robot navigating through a model of the aortic arch to deliver a catheter to several different arteries. Catheter delivery through the aortic arch is a critical step for endovascular surgeries to treat stroke and aneurysms. However, high tortuosity and highly variable anatomy of the aortic arch can make the navigation challenging. By leveraging the tip-growing robot architecture integrated with multiple LCE actuators and heaters, the robot was able to steer around a challenging Type Ill aortic arch and access the innominate artery (or brachiocephalic artery, BCT) as shown in FIG. 5(A). The robot diameter and length were 5 mm and 217 mm, respectively, and 4 LCE actuators (t_{LCE}=170 μm) and heaters are embedded into the robot 500. A catheter 510 with 2.06 mm outer diameter was attached at the tip of the robot 500, and was also used for controlling the robot length. A 3D-printed rigid introducer 505 that covers the base of the robot 500 is inserted into the model and fixed in position during navigation. The robot 500 can access other two arteries (LCC and LSB) by changing the introducer position and orientation and by activating different sets of heaters.

### Robot Application Example—Inspection Tasks

The disclosed technologies demonstrate the ability of an example robot to perform a mock inspection task inside a jet engine model. Inspection of complex machinery often relies on the use of a steerable endoscope equipped with a camera. These tools, however, can be limited in their ability to achieve multiple tight curvatures and safe interaction with environment. The example robots can steer with high curvature and can navigate the environment with minimal interaction, making them well-suited for such machine inspection tasks.

An example of the jet engine model is 3D-printed (for example, X1 Carbon, Bambu lab), and a 5 mm×5 mm camera (for example, 1000TVL, ¼″ CMOS) is integrated at the tip of the robot. Since the camera can move forward twice as fast as the robot, the camera wires are manually held at the base to keep the camera at the tip of the robot.

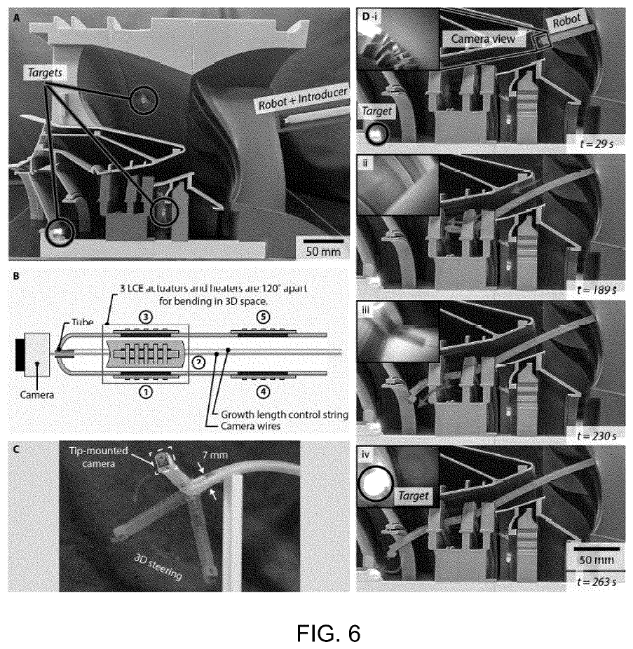

FIG. 6 shows an example application of a robot navigating a mock jet engine model and demonstrating inspection according to an embodiment of the present disclosure. Specifically, FIG. 6 shows the robot navigating to three selected target locations inside a 3D-printed jet engine model. FIG. 6(A) shows a side view of the model and the robot. The robot is covered by the rigid introducer. In this example, three inspection targets are located inside the engine model. FIG. 6(B) shows a schematic diagram of the robot used in this example. AS shown in this example, a camera is integrated at the tip, and three LCE actuators and heaters are embedded at the distal part of the robot for steering and inspecting in 3D space. FIG. 6(C) shows the example robot demonstrating 3D steering. As shown, three photos are overlaid. FIG. 6(D) shows video stills of the robot navigating the jet engine model and reaching the target. Insets of FIG. 6(D) show an example camera view.

To successfully reach these targets, the robot had to steer in both free spaces to reach the yellow target and through extremely tight spaces to reach green and red targets as shown in FIG. 6(A). In this example, the robot design is modified to include a tip-mounted camera and 3D steering capabilities needed for inspection as shown in FIG. 6(B). A small tube was attached at the tip of the robot, and a string for controlling the growth length was attached to the base of this tube. The camera is placed at the robot tip, and its electrical wires were fed through the tube, then through the robot body to the base. The disclosure integrates three sets of LCE actuators (170 μm thick) and heaters spaced equally around the robot circumference at the distal end of the robot as shown in FIG. 6(B) to enable scanning motions at the robot tip. However, the number of sets of LCE actuators and heaters is not limiting. FIG. 6(C) shows the overlaid photos of the robot steering in 3D space. The robot diameter is increased to 7 mm in this demonstration in order to achieve a higher bending stiffness to overcome the gravitational force from the mass of the robot and the camera at the tip. FIG. 6(D) shows the time series of the robot navigating through the jet engine model to reach the green target, and the insets show the camera view. As shown in FIG. 6(D)-ii, the example robot navigates through the tight spaces formed by the green curved blades and bends up. Since the size of rigid camera was comparable to the gap between the blades, the camera could get stuck as the robot grew. In these examples, the camera wires are gently pulled from the base to allow the camera to move freely. Once the example robot successfully passes through the sets of these green blades, it bends down to reach the target as shown in FIG. 6(D)-iii. Finally, the example robot utilizes its 3D steering capability to better inspect the target as shown in FIG. 6(D)-iv. In the examples, the example robot can reach all three targets by adjusting the introducer position and orientation. While this demonstration was performed in a miniaturized engine model, the example robot is expected to perform similarly at larger scales given that the steering performance is scale-independent if the example robot is isometrically scaled.

The disclosed technologies provide small-scale, steerable, and fully-soft growing robots driven by a functionalized robot skin integrated with LCE actuators and flexible heaters. By sandwiching and heat-welding TPU films with the functional components, robots from 3 to 7 mm in diameter were successfully manufactured. The example designs demonstrate large bending angles at multiple points along their body, even showing their ability to steer in 3D space. Using a simple model for the bending of the robot, the scaling analysis, along with extensive experimental characterization, shows how the bending angle is affected by the control inputs and design parameters. The example robot can demonstrate squeezing through a small gap 50% thinner than the robot diameter and navigating through tortuous environments. Furthermore, the example robot can illustrate its potential for use in various practical applications, such as catheter delivery within the aortic arch and inspection inside complex machinery, by integrating additional tools, such as a catheter or a camera. Through a series of demonstrations, the present disclosure shows how the steering of soft growing robots with LCE actuators can be effective for a wide range of applications that require small-scale steerable soft continuum robots. The example demonstrations are performed with visual feedback from the operator.

The disclosed technologies use pressure as one control input to modulate the bending angle, which was simple and effective for controlling the angle at the distal portion of the robot. However, controlling the pressure to achieve one bending angle could also affect the angles at other points along the robot body. By combining both pressure and temperature, the capabilities and performance of these robots are further improved. In particular, integrating temperature sensors enables closed-loop control of each LCE, which enables the bending angles at every point along the robot backbone to be fully decoupled. In addition, this hybrid control strategy can increase the range of achievable bending angles for a given robot design and ensure precise control of the robot shape.

Lastly, in some embodiments, the example robot design can be optimized for different scales or extended to enable even more complex robot shapes. In particular, further miniaturization of the robot (e.g., <2 mm in diameter) enables an even wider range of applications, especially in the medical setting. In order to achieve large bending angles at pressures above the growth pressure, smaller diameter robots require thinner skins and lower friction. To expand the complexity of achievable robot shapes, the number, shape, and placement of the LCE actuators can be optimized. In some examples, helical LCE actuators can be integrated to enable twisting.

As demonstrated herein, the disclosed robot design and manufacturing approach functionalizes a soft skin with actuators and heaters, while maintaining a thin form-factor, and enables new capabilities for steerable devices such as soft growing robots. The soft skin provided by the disclosed technologies can further be adapted for other various soft robotic systems, such as wearable haptic devices, soft grippers, and locomotive soft robots-all of which require thin, soft, and robust actuation.

### Robot Fabrication Example

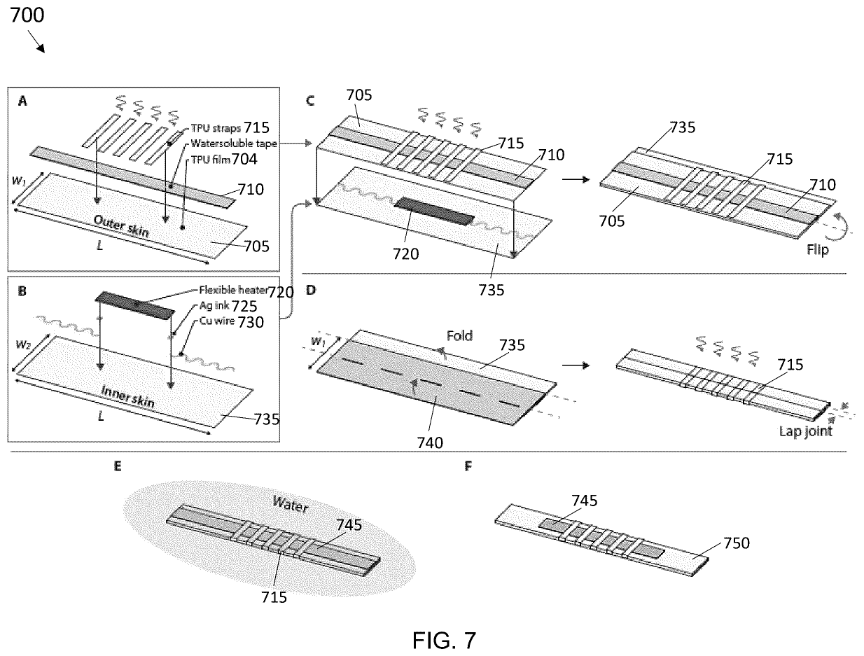

FIG. 7 shows an example fabrication process 700 for an LCE-driven steerable soft growing robot 750 according to an embodiment of the present disclosure. FIG. 7(A) shows an outer skin 705 with TPU straps 715 is manufactured by heat-pressing. FIG. 7(B) shows a flexible heater 720 and copper wires 730 are attached to the inner skin 735. FIG. 7(C) shows the outer skin 705 and inner skin 735 are heat-pressed, which encapsulated the heater 720. FIG. 7(D) shows the assembly is folded to form a tube with a lap joint and is heat-pressed. FIG. 7(E) shows water-soluble tapes 740 are dissolved. FIG. 7(F) shows LCE actuators 745 are attached to the tube.

Specifically, three layers of thermoplastic polyurethane (TPU) films (25 μm, BT-2085, American Polyfilm) are stacked, rolled into a cylinder with a lap joint, and heat-welded to manufacture the robot 750 as shown in FIG. 7. First, the outer skin 705 is manufactured by sandwiching a strip of water-soluble tape 710 between a rectangular TPU film 704 and TPU straps 715. The TPU films 704 and water soluble tapes 710 used in the process are laser-cut into the desired shapes. The stacked films were heat-pressed at 160° C. for 60 seconds as shown in FIG. 7(A) (DG16x24 GB, Fancier Studio). The water-soluble tape 710 is used to create a space between the TPU film 704 and straps 715 for the LCE actuator 745. The length of the rectangular film 705, L, is equal to the length of the robot 750, and the width of the film 705, w1, is equal to πD0, where DO is the robot diameter. Second, the inner skin 735 is prepared by attaching copper wires 730 (44 AWG, Remington Industries) onto the other rectangular TPU film 704 using a spray adhesive (6065, 3M). A small amount of either silver epoxy adhesive (8331D, MG Chemicals) or silver ink (CI-1036, Nagase ChemteX) is applied at the tip of the copper wires 730 through a stencil, providing a good electrical connection between the wire 730 and the heater 720. Then, the heaters 720 are placed on the TPU film 735 while making contact with the copper wires 730 as shown in FIG. 7B. The width of the inner skin 735 (w₂) is greater than w₁ in order to create a lap joint when forming the tube. The outer skin 705 and inner skin 735 are then placed on top of each other and heat-pressed at 138° C. for 60 seconds which allows for encapsulation of the heater 720 within two TPU layers 705, 735 as shown in FIG. 7(C). The assembly is then flipped upside down and a strip of water-soluble tape 740 with a fold line in the middle and width of w₁ is attached. This assembly is folded together to form a tube with a lap joint and is heat-pressed at 138° C. for 60 seconds as shown in FIG. 7(D). The water-soluble tapes 740 are then dissolved under flowing water and air-dried as shown in FIG. 7(E). LCE actuators 745 are inserted between the outer skin 705 and TPU straps 715 and are fixed at both ends onto the outer skin 705 using UV curable adhesive (SI 5240 with CL 32, Loctite) as shown in FIG. 7(F). Lastly, to turn a tube into an everting robot, one end of the tube is sealed and invert inside itself, and the other end is connected to a compressed air source using a Luer Lock connector and heatshrink. Depending on the application and tasks, a growth length control string, catheter, a wire or a tube were integrated at the tip of the robot. When only a growth length control string is integrated into the robot 750, an impulse sealer (FS-200) is used to heat-weld the tip of the robot 750 with the string. For integrating the catheter and the tube, UV curable adhesive (AA 3936, Loctite) is utilized. Dry lubricant (potato starch) is used inside the robot body to minimize the friction between the tail and the body during inversion and eversion.

### LCE Actuator Fabrication Example

4-(6-(acryloyloxy) hexyloxy)phenyl-4-(6-(acryloyloxy) hexyloxy)benzoate (C6BAPE, Chemfish, 97%), 2,2′-(ethylenedioxy) diethanethiol (EDDET; Sigma-Aldrich; 95%), pentaerythritol tetrakis(3-mercaptopropionate) (PETMP, Sigma-Aldrich, 95%), 2,4,6-Triallyloxy-1,3,5-triazine (TAC, Sigma-Aldrich, 97%), dipropylamine (DPA, Sigma-Aldrich, 98%), (2-hydroxyethoxy)-2-methyl-propiophenone (HHMP; Sigma-Aldrich; 98%), and the solvents are used as received without further purification.

To synthesize the LCE thin film, the disclosed technologies are used to dissolve the monomer C6BAPE (10.0 g) and TAC in toluene (3.1 g) and waited for 30 minutes at 85° C. to fully dissolve. Then, the crosslinker PETMP, chain extender EDDET, catalyst DPA solution (1 wt. % in toluene), and photo-initiator HHMP in the solution are sequentially added. The resulting mixture is stirred at 300 rpm for 2 minutes and vacuumed for 5 minutes. Next, the solution is poured into a glass mold and is placed in a dark environment for 24 hours for the first-stage crosslinking reaction to happen and followed by another 24 hours at 85° C. to evaporate the solvent. Finally, the loosely crosslinked LCE, applied a stretch of 2, is taken out and placed under UV (365 nm) for 60 minutes to get the monodomain LCE. Once fully cured, scissors are used to cut the LCE into the desired dimensions.

### PEDOT:PSS Flexible Heater Fabrication Example

The PEDOT:PSS solution is made by mixing PEDOT:PSS (for example, Clevios PH 1000, Heraeus), dimethyl sulfoxide (for example, from Sigma-Aldrich), and Triton X-100 (Sigma-Aldrich) in weight ratio of 92:5:3. Then the solution is mixed with deionized water (Sigma-Aldrich) in 1:1 weight ratio. 3 mL of the final PEDOT:PSS solution is drop-casted onto a glass slide (75 mm×50 mm) and is cured at 50° C. for 4 hours on a hot plate. The glass slide is cleaned with detergent and water, acetone (for example, from Sigma-Aldrich), and isopropyl alcohol (for example, from Sigma-Aldrich), and then dried in air before drop-casting. The cured PEDOT:PSS film is cut into desired the shapes with a knife. The film thickness is approximately 50 μm.

### Experimental Methods

In some embodiments, a pressure regulator (for example, QBX series, Proportion-Air) is used to control the pressure inside the robot, and a pressure sensor (for example, SSCDANN060PGAA5, Honeywell) is integrated at the robot base. For characterizing the steering angle, a power supply (for example, SPD3303X, Siglent) is used for applying electrical power to the heater during actuation. When measuring the quasi-static bending angle, a desired pressure is first set, and the heater is then turned on. The heater is left on until the bending angle reached its steady state. A webcam (for example, Logitech, 1080 HD) is used to capture photos, and the bending angles is photographically measured. A small spacer is placed between the robot and the ground at the base, which helps to minimize friction and heat loss to the ground.

The robot demonstration requires individual control over multiple heaters. To provide a constant current to the heaters, a current source (for example, LT3092, Analog Devices) commanded by the output voltage from a digital to analog converter (for example, DAC, MCP4725, SparkFun) is used. The DAC communicates via I2C communication with a microcontroller (for example, Uno, Arduino). To manage up to six heaters and communicate with multiple I2C devices, a multiplexer (for example, TCA9548A, Adafruit) is used. Please note, the number of heaters is not limiting.

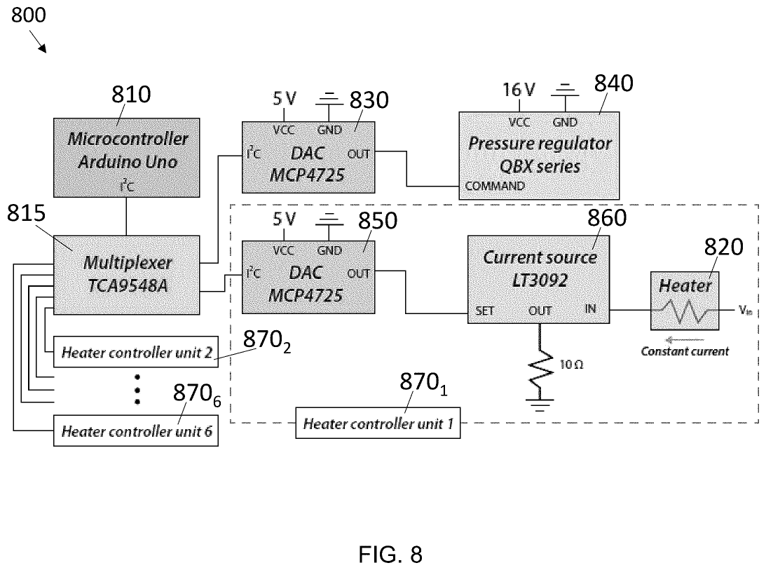

FIG. 8 shows a schematic diagram of a circuit 800 used for controlling a pressure inside the hollow interior and temperatures of a plurality of actuators according to an embodiment of the present technology. The circuit 800 is configured to determine one or more actuators from the plurality of actuators based on the desirable path, cause one or more heaters corresponding to the one or more actuators to provide respective temperatures to the one or more actuators so that the steerable device bends at respective one or more locations at respective steering angles based on the desirable path, and cause the pressure regulator to provide a desirable pressure inside the elongate body of the steerable device.

As shown in FIG. 8, the circuit 800 includes a microcontroller 810 configured to provide a first control signal to a first digital-analog converter (DAC) 830 coupled to the pressure regulator 840 and provide one or more second control signals to six heater controller units 870₁, 870₂, . . . , 870₆. corresponding to six heaters 820. Please note the number of the heater controller units is nonlimiting. The microcontroller 810 is further configured to transmit the first control signal and the one or more second control signals to a multiplexer 815. The multiplexer 815 is coupled to the microcontroller 810 and a plurality of heater controller units 870₁, 870₂, . . . , 870₆.

The multiplexer 815 is configured to receive the first control signal and the one or more second control signals from the microcontroller 810, transmit the first control signal to the first DAC 830, and transmit the one or more second control signals to the one or more heater controller units 870₁, 870₂, . . . , 870₆ corresponding to the one or more heaters 820.

Each of the heater controller units 870₁, 870₂, . . . , 870₆ comprises a second DAC 850 coupled to the multiplexer 815 and configured to receive a respective one of the one or more second control signals from the multiplexer 815, generate a respective setting signal based on the respective one of the one or more second control signals, transmit the respective setting signal to a current source 860. The current source 860 is coupled to the second DAC 850 and configured to generate a current based on the respective setting signal upon receipt from the second DAC 850 and apply the current to a respective heater 820.

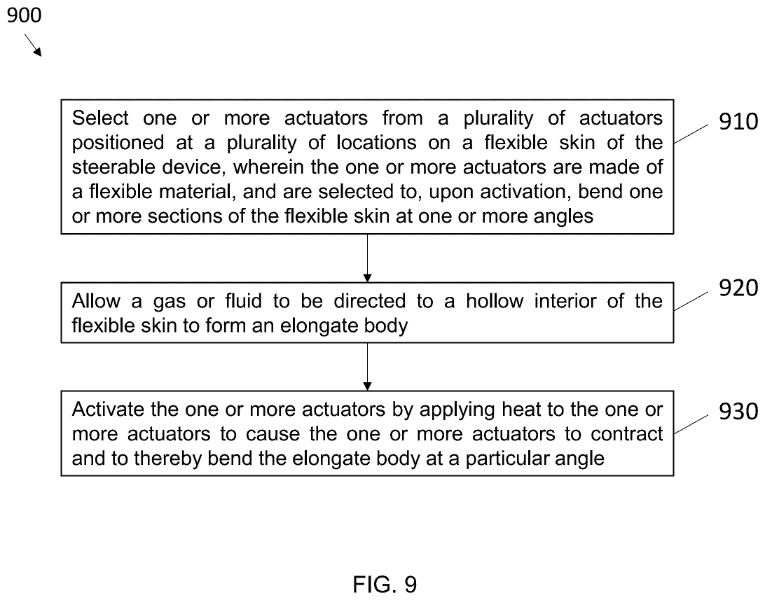

FIG. 9 shows a flowchart of implementing a method 900 of navigation a steerable device according to an embodiment of the present disclosure. An example of the steerable device is a soft growing robot as discussed above. At step 910, one or more actuators are selected from a plurality of actuators positioned at a plurality of locations on a flexible skin of the steerable device. The one or more actuators are made of a flexible material, and are selected to, upon activation, bend one or more sections of the flexible skin at one or more angles.

At step 920, a gas or a fluid is allowed to be directed to a hollow interior of the flexible skin to form an elongate body.

At step 930, the one or more actuators are activated by applying heat to the one or more actuators to cause the one or more actuators to contract and to thereby bend the elongate body at a particular angle.

In some embodiments, bending of the elongate body can be accomplished by any of the following, or a combination thereof: (1) changing the temperature to bend the elongate body at a larger angle (e.g., a higher temperature results in a higher angle), (2) keeping temperature constant (as long as it is above a certain temperature) and change pressure to change angle. Pressure can also be used to control the growth in the longitudinal direction.

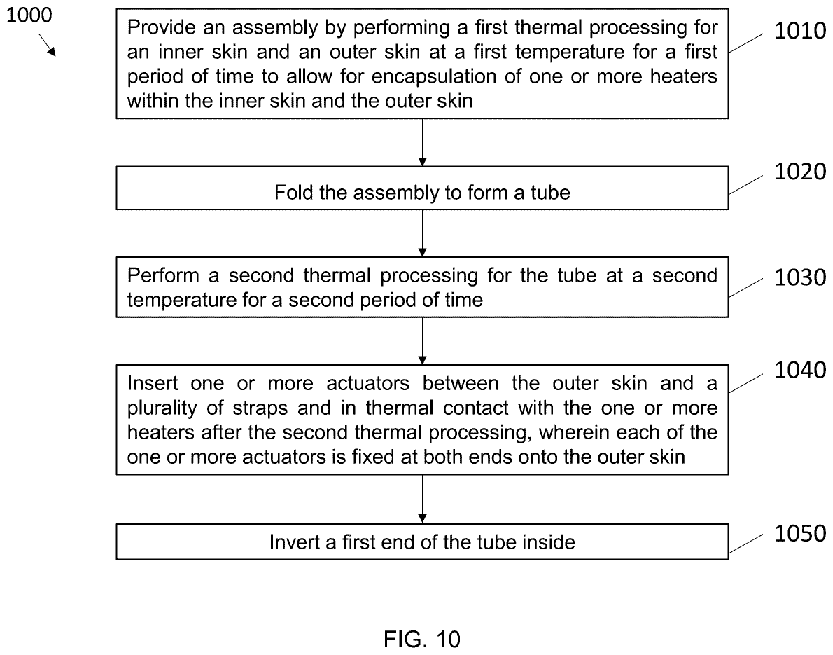

FIG. 10 shows a flowchart of implementing a method 1000 of manufacturing a steerable device according to an embodiment of the present disclosure. An example of the steerable device is a soft growing robot as discussed above. At step 1010, an assembly is provided by performing a first thermal processing for an inner skin and an outer skin at a first temperature for a first period of time to allow for encapsulation of one or more heaters within the inner skin and the outer skin. In some examples, the first temperature is 138° C. and the first period of time is 60 seconds. At step 1020, the assembly is folded to form a tube.

At step 1030, a second thermal processing is performed for the tube at a second temperature for a second period of time. In some examples, the second temperature is 138° C. and the second period of time is 60 seconds. At step 1040, one or more actuators are inserted between the outer skin and a plurality of straps and in thermal contact with the one or more heaters after the second thermal processing. Each of the one or more actuators is fixed at both ends onto the outer skin. At step 1050, a first end of the tube is inverted inside.

In some examples, a diameter of an elongate body of the steerable device is no greater than 5 mm. In some examples, the temperature of the respective actuator ranges between 45° C. and 65° C. In some examples, the respective steering angle increases when the pressure regulator reduces the pressure inside the elongate body of the steerable device, and the respective steering angle decreases when the pressure regulator increases the pressure inside the elongate body of the steerable device.

In some embodiment, the outer skin is prepared by sandwiching a strip of soluble tape between a first film and the plurality of straps. The soluble tape is a water-soluble tape configured to create a space between the first film and the plurality of straps. The first film and the strip of soluble tape are laser-cut into desired shapes.

A third thermal processing is performed for the outer skin at a third temperature for a third period of time. The inner skin is prepared by attaching a plurality of metal wires onto a second film and placing the one or more heaters on the second film and in contact with the plurality of metal wires. In some examples, the third temperature is 160° C. and the third period is 60 seconds. The first film is a rectangular film, and a length of the first film is equal to a length of the steerable device. The first film has a width equal to IT times a diameter of the steerable device.

The above description and drawings are illustrative and are not to be construed as limiting. Numerous specific details are described to provide a thorough understanding of the disclosure. However, in some instances, well-known details are not described in order to avoid obscuring the description. Further, various modifications may be made without deviating from the scope of the embodiments.

The use of examples anywhere in this specification including examples of any term discussed herein is illustrative only, and is not intended to further limit the scope and meaning of the disclosure or of any exemplified term. Likewise, the disclosure is not limited to various embodiments given in this specification.

While this patent document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Only a few examples and implementations are disclosed. Variations, modifications, and enhancements to the described examples and implementations and other implementations can be made based on what is disclosed.

### EXAMPLES

Example 1. A steerable device, comprising: an elongate body comprising a flexible skin that surrounds a hollow interior; a plurality of actuators positioned at a plurality of locations of the flexible skin, wherein each of the plurality of actuators includes a material that selectively contracts or expands in response to a change in temperature; and a plurality of thermal elements coupled to the plurality of actuators, each of the plurality of thermal elements operable to change a temperature of a respective actuator to cause the respective actuator to contract or expand, wherein the hollow interior is configured to receive a gas or a fluid to allow expansion of the flexible skin along a longitudinal direction, and wherein the elongate body is configured to be expanded along the longitudinal direction in response to a flow of the gas or the fluid inside the hollow interior of the flexible skin and to be bent at one or more angles in response to at least a temperature change of one or more of the plurality of actuators.
Example 2. The steerable device of any one or more examples disclosed herein, wherein at least one of the plurality of actuators comprises a liquid crystal elastomer (LCE) material or a shape memory polymer.
Example 3. The steerable device of any one or more examples disclosed herein, wherein each of the plurality of actuators is made of a flexible material and has an original size at a first temperature, and wherein each of the plurality of actuators is configured to contract in response to receiving heat that increases the actuator's temperature to a second temperature, and to expand back to the original size upon removal of the heat. For example, if the actuator is made of LCE, removing the heat allows the actuator to return to its original size. It is understood, however, that with some materials, the actuator may not return back to its original size.
Example 4. The steerable device of any one or more examples disclosed herein, wherein contraction of each of the plurality of actuators is based on an amount of heat received by the actuator such that a larger amount of heat causes a larger bending angle.
Example 5. The steerable device of any one or more examples disclosed herein, wherein the plurality of actuators includes three or more actuators that are arranged radially on the flexible skin to allow the steerable device to bend at the one or more angles in three-dimensions.
Example 6. The steerable device of any one or more examples disclosed herein, wherein the plurality of actuators includes two or more actuators that are arranged substantially back-to-back longitudinally on the flexible skin to effectuate a varying amount of bending angle by imparting a temperature change to fewer or more of the plurality of actuators.
Example 7. The steerable device of any one or more examples disclosed herein, wherein a first bending angle is obtained by imparting temperature increase to a first of the two or more actuators, and a second bending angle, which is larger than the first bending angle, is obtained by imparting temperature increase to the first and to a second of the plurality of actuators.
Example 8. The steerable device of any one or more examples disclosed herein, wherein the plurality of actuators includes two or more actuators that are arranged longitudinally on the flexible skin at two or more longitudinal locations separated from each other by a particular distance to allow the steerable device to bend at the two or more longitudinal locations.
Example 9. The steerable device of any one or more examples disclosed herein, wherein the flexible skin is configured to be tucked in or folded inside the hollow interior and to expand in the longitudinal direction in response to receiving the gas or fluid, or an increase in a pressure of the gas or fluid.
Example 10. The steerable device of any one or more examples disclosed herein, further including a flexible insert within the hollow body of the flexible skin configured to inhibit expansion of the flexible skin in the longitudinal direction beyond one or more predetermined lengths.
Example 11. The steerable device of any one or more examples disclosed herein, wherein the flexible insert is one of a string, a catheter, a wire, or a tube.
Example 12. The steerable device of any one or more examples disclosed herein, wherein the elongate body of the steerable device is deformable so that the steerable device can pass through a gap that is smaller than a diameter of the steerable device.
Example 13. The steerable device of any one or more examples disclosed herein, wherein a thickness of each of the plurality of actuators ranges between 80 and 230 microns (μm).
Example 14. The steerable device of any one or more examples disclosed herein, further comprising: a pressure sensor configured to measure a pressure of the gas or fluid; and a pressure regulator configured to control the pressure inside the elongate body.
Example 15. The steerable device of any one or more examples disclosed herein, wherein a length of the steerable device and a plurality of angles are changeable in response to a combination of a change in a gas or fluid pressure inside the hollow body and a temperature change at the plurality of actuators.
Example 16. The steerable device of any one or more examples disclosed herein, further comprising a camera mounted at the tip of the of the elongate body and configured to capture one or more images around the steerable device.
Example 17. A method of navigating a steerable device, comprising: selecting one or more actuators from a plurality of actuators positioned at a plurality of locations on a flexible skin of the steerable device, wherein the one or more actuators are made of a flexible material, and are selected to, upon activation, bend one or more sections of the flexible skin at one or more angles; allowing a gas or a fluid to be directed to a hollow interior of the flexible skin to form an elongate body; and activating the one or more actuators by applying heat to the one or more actuators to cause the one or more actuators to contract and to thereby bend the elongate body at a particular angle.
Example 18. The method of any one or more examples disclosed herein, comprising causing the elongate body to bend at a larger angle by increasing the heat imparted to one or more of the actuators or by activating additional actuators that are directly adjacent to the one or more actuators.
Example 19. The method of any one or more examples disclosed herein, comprising controlling a movement of the elongate body in a longitudinal direction by changing a pressure of the gas or fluid that flows through the hollow interior.
Example 20. The method of any one or more examples disclosed herein, comprising deactivating the one or more actuators by removing or reducing an amount of heat that is applied thereto to reduce or eliminate a bent in the elongate body.
Example 21. The method of any one or more examples disclosed herein, comprising directing a movement of the elongate body along a particular path by one or more of the following: (a) sequentially activating or deactivating a subset of the plurality of activators that are positioned around a periphery of the elongate body, (b) sequentially or simultaneously activating or deactivating a subset of the plurality of activators that are positioned in a longitudinal direction on the elongate body, or (c) adjusting a pressure of the gas or fluid that is directed to the hollow interior.
Example 22. The method of any one or more examples disclosed herein, comprising using a flexible insert internal to the elongate body to prevent elongation of the elongate body beyond a particular length.
Example 23. The method of any one or more examples disclosed herein, wherein the flexible skin is initially tucked in or folded inside the hollow interior, the steerable device is inserted in a cavity, and thereafter, the gas or the fluid is directed to the hollow interior to form the elongate body.
Example 24. The method of any one or more examples disclosed herein, comprising applying the heat to the one or more actuators to maintain a temperature of the one or more actuators at a value above a predetermined threshold, and changing a pressure of the gas or the fluid to effectuate bending of the elongate body.
Example 25. A method of manufacturing a steerable device, comprising: providing an assembly by performing a first thermal processing for an inner skin and an outer skin at a first temperature for a first period of time to allow for encapsulation of one or more heaters within the inner skin and the outer skin; folding the assembly to form a tube; performing a second thermal processing for the tube at a second temperature for a second period of time; inserting one or more actuators between the outer skin and a plurality of straps and in thermal contact with the one or more heaters after the second thermal processing, wherein each of the one or more actuators is fixed at both ends onto the outer skin; and inverting a first end of the tube inside.

1. A steerable device, comprising:
an elongate body comprising a flexible skin that surrounds a hollow interior;
a plurality of actuators positioned at a plurality of locations of the flexible skin, wherein each of the plurality of actuators includes a material that selectively contracts or expands in response to a change in temperature; and
a plurality of thermal elements coupled to the plurality of actuators, each of the plurality of thermal elements operable to change a temperature of a respective actuator to cause the respective actuator to contract or expand,
wherein the hollow interior is configured to receive a gas or fluid to allow expansion of the flexible skin along a longitudinal direction, and
wherein the elongate body is configured to be expanded along the longitudinal direction in response to a flow of the gas or fluid inside the hollow interior of the flexible skin and to be bent at one or more angles in response to at least a temperature change of one or more of the plurality of actuators.

2. The steerable device of claim 1, wherein at least one of the plurality of actuators comprises a liquid crystal elastomer (LCE) material or a shape memory polymer.

3. The steerable device of claim 1, wherein each of the plurality of actuators is made of a flexible material and has an original size at a first temperature, and wherein each of the plurality of actuators is configured to contract in response to receiving heat that increases the actuator's temperature to a second temperature, and to expand back to the original size upon removal of the heat.

4. The steerable device of claim 3, wherein contraction of each of the plurality of actuators is based on an amount of heat received by the actuator such that a larger amount of heat causes a larger bending angle.

5. The steerable device of claim 1, wherein the plurality of actuators includes three or more actuators that are arranged radially on the flexible skin to allow the steerable device to bend at the one or more angles in three-dimensions.

6. The steerable device of claim 1, wherein the plurality of actuators includes two or more actuators that are arranged substantially back-to-back longitudinally on the flexible skin to effectuate a varying amount of bending angle by imparting a temperature change to fewer or more of the plurality of actuators.

7. The steerable device of claim 6, wherein a first bending angle is obtained by imparting temperature increase to a first of the two or more actuators, and a second bending angle, which is larger than the first bending angle, is obtained by imparting temperature increase to the first and to a second of the plurality of actuators.

8. The steerable device of claim 1, wherein the plurality of actuators includes two or more actuators that are arranged longitudinally on the flexible skin at two or more longitudinal locations separated from each other by a particular distance to allow the steerable device to bend at the two or more longitudinal locations.

9. The steerable device of claim 1, wherein the flexible skin is configured to be tucked in or folded inside the hollow interior and to expand in the longitudinal direction in response to receiving the gas or fluid, or an increase in a pressure of the gas or fluid.

10. The steerable device of claim 1, further including a flexible insert within the hollow body of the flexible skin configured to inhibit expansion of the flexible skin in the longitudinal direction beyond one or more predetermined lengths.

11. The steerable device of claim 10, wherein the flexible insert is one of a string, a catheter, a wire or a tube.

12. The steerable device of claim 1, wherein the elongate body of the steerable device is deformable so that the steerable device can pass through a gap that is smaller than a diameter of the steerable device.

13. The steerable device of claim 1, wherein a thickness of each of the plurality of actuators ranges between 80 and 230 microns (μm).

14. The steerable device of claim 1, further comprising:
a pressure sensor configured to measure a pressure of the gas or fluid; and
a pressure regulator configured to control the pressure inside the elongate body.

15. The steerable device of claim 1, wherein a length of the steerable device and a plurality of angles are changeable in response to a combination of a change in a gas or fluid pressure inside the hollow body and a temperature change at the plurality of actuators.

16. The steerable device of claim 1, further comprising a camera mounted at the tip of the of the elongate body and configured to capture one or more images around the steerable device.

17. A method of navigating a steerable device, comprising:
selecting one or more actuators from a plurality of actuators positioned at a plurality of locations on a flexible skin of the steerable device, wherein the one or more actuators are made of a flexible material, and are selected to, upon activation, bend one or more sections of the flexible skin at one or more angles;
allowing a gas or fluid to be directed to a hollow interior of the flexible skin to form an elongate body; and
activating the one or more actuators by applying heat to the one or more actuators to cause the one or more actuators to contract and to thereby bend the elongate body at a particular angle.

18. The method of claim 17, comprising causing the elongate body to bend at a larger angle by increasing the heat imparted to one or more of the actuators or by activating additional actuators that are directly adjacent to the one or more actuators.

19. The method of claim 17, comprising controlling a movement of the elongate body in a longitudinal direction by changing a pressure of the gas or fluid that flows through the hollow interior.

20. The method of claim 17, comprising deactivating the one or more actuators by removing or reducing an amount of heat that is applied thereto to reduce or eliminate a bent in the elongate body.

21. The method of claim 17, comprising directing a movement of the elongate body along a particular path by one or more of the following:
(a) sequentially activating or deactivating a subset of the plurality of activators that are positioned around a periphery of the elongate body,
(b) sequentially or simultaneously activating or deactivating a subset of the plurality of activators that are positioned in a longitudinal direction on the elongate body, or
(c) adjusting a pressure of the gas or fluid that is directed to the hollow interior.

22. The method of claim 17, comprising using a flexible insert internal to the elongate body to prevent elongation of the elongate body beyond a particular length.

23. The method of claim 17, wherein the flexible skin is initially tucked in or folded inside the hollow interior, the steerable device is inserted in a cavity, and thereafter, the gas or fluid is directed to the hollow interior to form the elongate body.

24. The method of claim 17, comprising applying the heat to the one or more actuators to maintain a temperature of the one or more actuators at a value above a predetermined threshold, and changing a pressure of the gas or the fluid to effectuate bending of the elongate body.

25. A method of manufacturing a steerable device, comprising:
providing an assembly by performing a first thermal processing for an inner skin and an outer skin at a first temperature for a first period of time to allow for encapsulation of one or more heaters within the inner skin and the outer skin;
folding the assembly to form a tube;
performing a second thermal processing for the tube at a second temperature for a second period of time;
inserting one or more actuators between the outer skin and a plurality of straps and in thermal contact with the one or more heaters after the second thermal processing, wherein each of the one or more actuators is fixed at both ends onto the outer skin; and
inverting a first end of the tube inside the tube.
